(19) United States
(12) Patent Application Publication
(10) Pub. No.: US 2025/0179262 A1
(43) Pub. Date: Jun. 5, 2025

(54) DEPOLYMERIZATION OF POLYURETHANES: REGENERATION OF ISOCYANATES VIA CHEMICAL RECYCLING

(71) Applicants: Mridula NANDI, Newark, DE (US); Robert M. O'DEA, Newark, DE (US); Thomas H. EPPS, Bear, DE (US); LaShanda T.J. KORLEY, Middletown, DE (US)

(72) Inventors: Mridula NANDI, Newark, DE (US); Robert M. O'DEA, Newark, DE (US); Thomas H. EPPS, Bear, DE (US); LaShanda T.J. KORLEY, Middletown, DE (US)

(73) Assignee: University of Delaware, Newark, DE (US)

(21) Appl. No.: 18/845,271

(22) PCT Filed: Mar. 9, 2023
(86) PCT No.: PCT/US2023/014905

§ 371 Date: Sep. 9, 2024

Related U.S. Application Data
(60) Provisional application No. 63/318,123, filed on Mar. 9, 2022.

(51) Int. Cl. C08J 11/22; C07C 263/20; C08G 18/81

(52) U.S. Cl. CPC C08J 11/22; C07C 263/20; C08G 18/8125; C08J 2375/04

(57) ABSTRACT

The present invention discloses a one-step polyurethane (PU) depolymerization to recover isocyanates and regenerate both thermoplastic and thermoset PUs. The method uses a halide-containing boron compound to depolymerize PUs directly to isocyanates under mild conditions (e.g., <100° C. in toluene). Also, disclosed herein is depolymerization of both model and commercially available PUs and regeneration of PUs using recovered isocyanates, with molecular weight distributions and thermomechanical properties that are equivalent to virgin PUs with yields of up to 50%.

### CROSS-REFERENCE TO RELATED APPLICATION

This application claims priority to U.S. Provisional Patent Application No. 63/318,123, filed Mar. 9, 2022, the entire disclosure of which is incorporated herein by reference for all purposes.

### STATEMENT REGARDING FEDERALLY SPONSORED RESEARCH

This invention was made with government support under Grant Nos. NSF GCR 1934887 and NSF DMR 2004682 awarded by the National Science Foundation. The government has certain rights in the invention.

### BACKGROUND OF THE INVENTION

Polyurethanes (PU) are a ubiquitous class of polymers because they offer a wide range of accessible thermomechanical properties that can be tailored through the choice of monomers and additives used in manufacturing. PUs rank sixth in overall polymer production, and the global market is projected to grow from $70.7B in 2020 to $94.6B by 2028 at a compound annual growth rate (CAGR) of 3.8%. Conventional PUs are polymerized from isocyanates, polyols/macrodiols, and a low molecular weight diol/chain extender. The production of PUs results in environmental and human health concerns because most common diisocyanates for PU manufacturing, such as methylene diphenyl diisocyanate (MDI) and toluene diisocyanate (TDI), are classified as carcinogenic, mutagenic, and reprotoxic substances. Exposure to these compounds has been linked to asthma, dermatitis, conjunctivitis, respiratory disorders, and acute poisoning. Additionally, isocyanates are prepared by reacting amines with phosgene, a highly toxic substance. Despite the hazards associated with their production, PUs are versatile materials that are used in everyday products such as footwear, refrigerators, foams, mattresses, carpets, furniture, adhesives, coatings, composites, airplanes, windmills, and many others.

The widespread use of PU materials has led to the generation of a large amount of end-of-life PU and post-production scraps, a majority of which are landfilled or incinerated, and recycling these waste streams has been challenging. Presently, PU recycling strategies can be categorized as mechanical or thermochemical recycling. Mechanical recycling is the most common approach used to recycle thermoplastic PU and involves grinding solid PU waste into granules that then are rebound into PU foams or used as filler in new products. This technique typically degrades PUs and results in materials with inferior thermomechanical properties in comparison to virgin PUs, and additives or compatibilizers often are added to the recyclate to facilitate blending with virgin polymer. However, when it comes to thermoset PUs, mechanical recycling fails due to the network structure of the polymer.

In contrast to mechanical methods, thermochemical recycling involves deconstruction of the PU waste into monomers or other small molecules. The most common PU chemical recycling methods are hydrolysis, aminolysis, hydrogenolysis, and glycolysis. In hydrolysis, PUs are reacted with steam at elevated temperature (>200° C.) in presence of a basic catalyst, resulting in the formation of amines, polyols, and carbon dioxide. For aminolysis, deconstruction is carried out using ammonia or aliphatic amines, and amines, polyols, ureas, and carbamates are generated. Although this process involves milder reaction conditions in comparison to other chemical recycling processes, complicated separations steps hinder its industrial application. More recently, catalytic hydrogenolysis of PU wastes has emerged as a chemical recycling technique that employs external hydrogen and transition-metal catalysts, especially transition-metal pincer complexes, in combination with a base to deconstruct PU materials into the corresponding diamine and polyol fractions. Finally, in glycolysis, PU wastes are deconstructed by reacting with a diol or triol (e.g., ethylene glycol or glycerol) in the temperature range 180° C.-240° C. to form polyols that can be incorporated into new PU materials. Of these chemical recycling methods, only glycolysis is applied on an industrial scale, and none of the aforementioned techniques enable the recovery of isocyanates, a key component for PU synthesis.

Hence, there is a need for a robust strategy to recover isocyanates from PU deconstruction and to thereby reduce the dependence on petrochemical feedstocks, especially highly hazardous compounds such as phosgene, while increasing the circularity of PU materials.

### SUMMARY OF THE INVENTION

Disclosed herein is a method for the depolymerization of polyurethane materials that enables the recovery of isocyanates and regeneration of PU materials using an organoboron compound as the depolymerization agent under mild reaction conditions (FIG. 1A). Laboratory-made and commercially sourced thermoset and thermoplastic polyurethanes comprised of aromatic isocyanates have been depolymerized using the technique, as disclosed hereinbelow, and the recovered isocyanates have been used to regenerate new PU materials with molecular weights and thermomechanical properties that are equivalent to virgin materials.

In an aspect of the invention, a method for depolymerizing a polyurethane to provide a regenerated isocyanate compound in a single step is provided. The method includes contacting the polyurethane with a depolymerization agent in the presence of a solvent and a Lewis base. The depolymerization agent may include a halide-containing boron compound, and the regenerated isocyanate mixture may comprise a mixture of a regenerated isocyanate compound and at least one of an adduct of a polyol and the halide-containing boron compound, an adduct of a chain extender and the halide-containing boron compound, a residual amount of the Lewis base, or an acid-base adduct.

In an embodiment, the method may be used for depolymerizing a thermoplastic polyurethane or a thermoset polyurethane, as represented by the following structures:

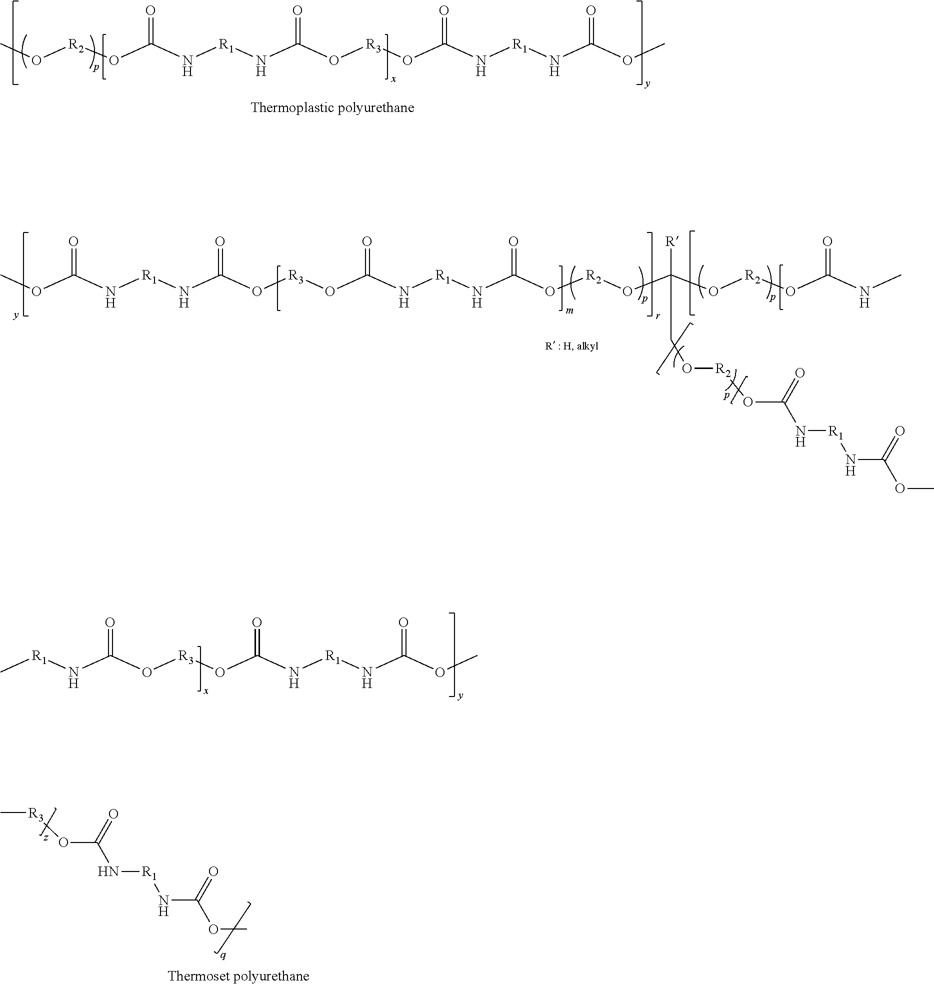

wherein:
R′ is H or a C1-C10 alkyl group;
each R₁ is independently:
   (i) an unsubstituted or a substituted alkylene group having 2 to 75 carbon atoms where one or more carbon atoms in the alkylene group may be substituted by oxygen, nitrogen or sulfur atoms with the proviso that only carbon atoms are directly bonded to each of the two NH groups in the polyurethane backbone; or
   (ii) an unsubstituted or a substituted aromatic-containing group having 6 to 75 carbon atoms; or
   (iii) an unsubstituted or a substituted heteroaromatic-containing group having 4 to 75 carbon atoms; or
   (iv) an unsubstituted or a substituted cycloalkyl-containing group having 4 to 75 carbon atoms; or
   (v) an unsubstituted or a substituted heterocycloalkyl-containing group having 4 to 75 carbon atoms; or
   (vi) combinations thereof;
R₂ and R₃ are independently selected from an alkylene group having 2-75 carbon atoms, where:
one of R₂ or R₃ is an alkylene group having 2 to 5 carbon atoms, and the other of R₂ or R₃ is an oligomeric or a polymeric segment having 6-75 carbon atoms, and
one or more carbon atoms is optionally substituted by oxygen, nitrogen, or sulfur atoms with the proviso that only carbon atoms are directly bonded to each of the end oxygen atoms in the polyurethane backbone;
X, y, z, m, n, and r, each independently is 1 to 200, and p is 3 to 100, and
wherein the depolymerization agent is present in an amount of 0.3 to 3 molar equivalents with respect to the urethane linkages.

In an embodiment of the method as disclosed hereinabove, the halide-containing boron compound is selected from a halo-organo-borane compound or a boron trihalide. In an embodiment, the halo-organo-borane compound may comprise one or more of β-chlorocatecholborane, β-bromocatecholborane, β-fluorocatecholborane, and β-iodocatecholborane.

In another embodiment of the method, the Lewis base may comprise one or more of triethylamine, pyridine, dimethylaminopyridine, triethylenediamine, diisopropylethylamine, other trialkylamines, 1,8-diazabicyclo [5.4.0]undec-7-ene (DBU), 1,5-diazabicyclo[4.3.0]non-5-ene (DBN), amidine derivatives, lithium diisopropylamide, and quinuclidine. In an embodiment, the solvent may comprise one or more of toluene, hexane, heptane, pentane, benzene, and cyclohexane.

In an embodiment of the method, the depolymerization is carried out at a temperature in a range of 20° C. to 150° C., in an inert environment comprising one or more of nitrogen (N₂), argon, helium, and dry air.

In another embodiment of the method, the polyol may comprise poly(tetramethylene oxide), polyethylene glycol, polyethylene glycol trimethylolpropane triether, polypropylene glycol, or combinations thereof, and the chain extender may comprise 1,4-butanediol, glycerol, ethylene glycol, 1,6-hexanediol, or combinations thereof.

In yet another embodiment of the method, the method may further comprise isolating recovered isocyanate from the regenerated isocyanate mixture by treatment of the regenerated isocyanate mixture with at least one of chromatography, distillation (e.g., as vacuum distillation), differential extraction, or crystallization.

In another aspect of the invention, disclosed herein is a method of forming a polyurethane comprising reacting the isolated recovered isocyanate with one or more polyols and/or chain extenders in the presence of a polymerization catalyst, and, an optional solvent. In an embodiment, the polymerization catalyst may comprise a tin-containing catalyst or an amine catalyst. In an embodiment, the tin-containing catalyst may comprise dibutyltin dilaurate, dioctyltin dilaurate, dicotyl carboxylate, stannous octoate, or mixtures thereof, and the amine catalyst may comprise 1,4-diazabicyclo[2.2.2]octane (DABCO), dimethylethanolamine, triethylenediamine, or mixtures thereof.

Another aspect of the invention disclosed herein is a composition comprising a polyurethane prepared from the isolated recovered isocyanate and a halide-containing boron compound. In an embodiment, the composition may further comprise a solvent. In another embodiment, the composition may further comprise one or more of a virgin polyurethane, an inert filler, and an additive. In an embodiment, the composition may have a bio-based content in the range of 20 to 100%, according to ASTM-D6866.

Another aspect of the invention is an article comprising the composition as disclosed hereinabove.

Another aspect of the invention disclosed herein is a method comprising providing a regenerated isocyanate mixture obtained by the method for depolymerizing a polyurethane, as disclosed hereinabove; purifying the regenerated isocyanate mixture compound by at least one of chromatography, distillation (e.g., as vacuum distillation), differential extraction, or crystallization to obtain an isolated recovered isocyanate compound; and forming a recycled polyurethane by reacting the recovered isocyanate compound with one or more polyols in the presence of a catalyst, wherein the one or more polyols comprises a recovered polyol, a bio-derived polyol, a petroleum-based polyol, or a mixture thereof.

### BRIEF DESCRIPTION OF THE DRAWINGS

FIG. 1A shows an exemplary one-step depolymerization of polyurethanes and regeneration of isocyanates and repolymerization according to embodiments of the present invention.

FIG. 1B shows generalized chemical structures of a thermoplastic polyurethane and a thermoset polyurethane.

FIG. 2A shows a schematic illustrating synthesis of polyurethanes by varying the diisocyanate and chain extender.

FIG. 2B shows a schematic illustrating an exemplary depolymerization reaction of a lab-synthesized hexamethylene diisocyanate (HDI)-based polyurethane (1.56 g) in the presence of β-chlorocatecholborane (0.52 g), triethylamine (0.7 mL) and toluene (10 mL) carried out at a temperature of 85° C., according to an embodiment of the present invention.

FIG. 2C shows an ATR-FTIR spectra of an exemplary HDI-based polyurethane (solid line) and of the product mixture after depolymerization reaction (dashed line) shown in FIG. 2B.

FIG. 2D shows analysis of the product mixture of FIG. 2B by gas chromatography-mass spectrometry (GC-MS).

FIG. 3A shows ATR-FTIR spectra of a model thermoplastic polyurethane

(PU1) before depolymerization, after depolymerization, after isocyanate purification, and after repolymerization.

FIG. 3B shows ATR-FTIR spectra of the commercial thermoplastic polyurethane (CPU1) before depolymerization, after depolymerization, after isocyanate purification, and after repolymerization. Spectra are offset vertically for clarity.

FIG. 4A shows gel permeation chromatography (GPC) chromatograms of thermoplastic polyurethanes: lab-synthesized (PU1) and commercially obtained (CPU1).

FIG. 4B show differential scanning calorimetry (DSC) thermograms (exotherm up) of lab synthesized thermoplastic polyurethane (PU1), and commercially obtained thermoplastic polyurethane (CPU1).

FIGS. 5A-5C show nuclear magnetic resonance (NMR) spectra of recovered isocyanates from lab-synthesized thermoplastic polyurethane (PU1), and commercially obtained thermoplastic polyurethane (CPU1), thermoset polyurethane (PU2) and commercially-obtained thermoset polyurethane (CPU2), and pure MDI and toluene diisocyanate (TDI) for reference.

FIGS. 6A-6B show (A): ATR-FTIR spectra of the model thermoset (PU2) before depolymerization (black), after depolymerization, after isocyanate purification, and after repolymerization; and (B): ATR-FTIR spectra of the commercial thermoset (CPU2) before depolymerization, after depolymerization, after isocyanate purification (blue), and after repolymerization. Spectra are offset vertically for clarity.

FIG. 7 shows GPC chromatograms of repolymerized thermoset polyurethanes from lab-synthesized thermoset polyurethane (PU2) and commercially-obtained thermoset polyurethane (CPU2).

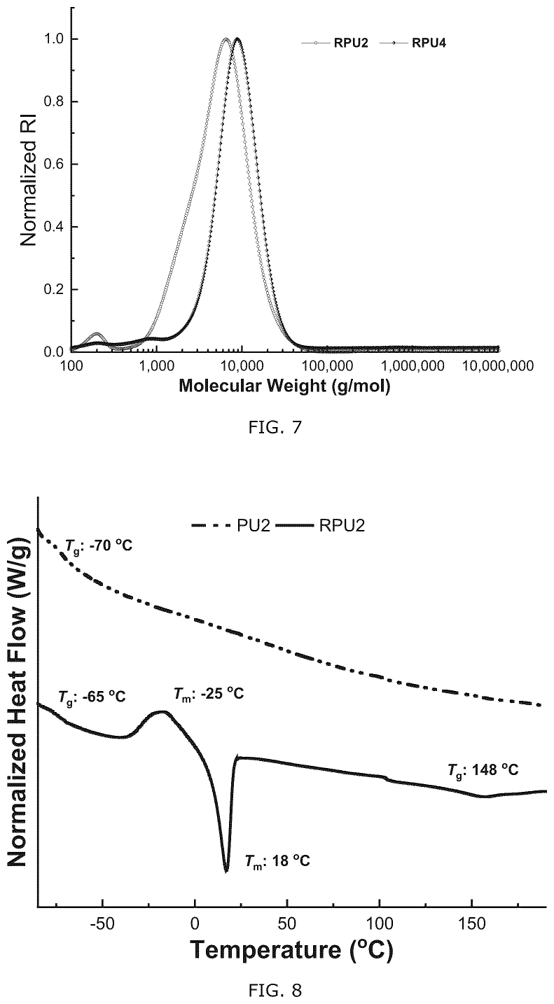

FIG. 8 shows DSC thermograms (exotherm up) of starting thermoset polyurethane (PU2) and resynthesized polyurethane (RPU2).

FIGS. 9A-9B show: (A): GPC chromatograms of remade PUs prepared from isolated MDI (RPU1, RPU3) where molecular weights were calculated relative to a series of poly(methyl methacrylate) standards; and (B): DSC curves (exotherm up) of resynthesized polymers RPU1 and RPU3. Curves are offset vertically for clarity.

FIGS. 10A-10B show (A): an ATR-FTIR spectrum of a thermoplastic polyurethane (PU1) after depolymerization using boron trichloride as a depolymerization agent; and (B): a nuclear magnetic resonance (NMR) spectrum of the product mixture after depolymerization reaction showing signals from isocyanates, polyol, and chain extender.

### DETAILED DESCRIPTION OF THE INVENTION

As used herein the term “virgin polyurethane” refers to polyurethanes produced from isocyanate(s) that are directly derived from either petrochemical feedstocks, like natural gas or crude oil, or plant-based biomass feedstocks.

As used herein, the term “alkylene group” refers to a saturated aliphatic hydrocarbon group such as an ethyl group, an n-propyl group, an isopropyl group, an n-butyl group, a sec-butyl group, or a tert-butyl group, and the alkyl group may have a substituent or no substituent. The term “substituted alkylene group” refers to an alkyl group bonded to a substituent; the additional substituent is not particularly limited. Examples of the additional substituent include an alkyl group, a halogen, an aryl group, and a heteroaryl group, and the same holds true in the description below. An alkyl group substituted with a halogen is also referred to as a haloalkyl group. The number of carbon atoms in the alkylene group is not particularly limited, and is preferably in the range of 2 to 75, or 3 to 50, or 5 to 35, or 5 to 25, or 5 to 20, or 5-10.

As used herein, the term “cycloalkyl-containing group” refers to an alkylene group containing a saturated alicyclic hydrocarbon moiety such as a cyclobutyl group, a cyclopentyl group, a cyclohexyl group, a cycloheptyl group, a norbornyl group, or an adamantyl group. The cycloalkyl-containing group may have a substituent or no substituent. The number of carbon atoms in the cycloalkyl-containing group is not particularly limited, and is preferably in the range of 4 to 75, or 3 to 50, or 5 to 35, or 5 to 25, or 5 to 20.

As used herein, the term “heterocycloalkyl-containing group” refers to an alkylene group containing an aliphatic ring having at least one atom other than carbon (such as N, O and/or S) in the ring, such as a piperazine ring, a pyrrolidine ring, a pyrrolidone ring, an azetidine ring, a morpholine ring, a dioxane ring, a tetrahydrofuran ring, an oxirane ring, a pyran ring, a piperidine ring, or a cyclic amide, and the heterocycloalkyl-containing group may have a substituent or no substituent. The number of carbon atoms in the heterocycloalkyl-containing group is not particularly limited, and is preferably in the range of 4 to 75, or 4 to 60, or 5 to 50, or 5 to 35, or 5 to 25, or 5 to 20.

As used herein, the term “aromatic-containing group” refers to an alkylene group containing a cyclic aromatic group, such as a phenyl group, a biphenyl group, a terphenyl group, a naphthyl group, a fluorenyl group, a benzofluorenyl group, a dibenzofluorenyl group, a phenanthryl group, an anthracenyl group, a benzophenanthryl group, a benzoanthracenyl group, a chrysenyl group, a pyrenyl group, a fluoranthenyl group, a triphenylenyl group, a benzofluoranthenyl group, a dibenzoanthracenyl group, a perylenyl group, or a helicenyl group. Among these groups, a phenyl group, a biphenyl group, a terphenyl group, a naphthyl group, a fluorenyl group, a phenanthryl group, an anthracenyl group, a pyrenyl group, a fluoranthenyl group, and a triphenylenyl group are preferable. The aromatic-containing group may have a substituent or no substituent. The number of carbon atoms in the aromatic-containing group is not particularly limited, and is preferably in the range of 6 to 75, or 6 to 50, or 6 to 35, or 6 to 25, or 6 to 20.

In a substituted phenyl group having two adjacent carbon atoms each having a substituent, the substituents may form a ring structure. The resulting group may correspond to any one or more of a “substituted phenyl group”, an “aryl group having a structure in which two or more rings are condensed”, and a “heteroaryl group having a structure in which two or more rings are condensed” depending on the structure.

As used herein, the term “heteroaromatic-containing group” refers to an alkylene group containing a cyclic aromatic group having one or a plurality of atoms other than carbon (such as N, O and/or S) in the ring, such as a pyridyl group, a furanyl group, a thiophenyl group, a quinolinyl group, an isoquinolinyl group, a pyrazinyl group, a pyrimidyl group, a pyridazinyl group. The heteroaryl-containing group may have a substituent or no substituent. The number of carbon atoms in the heteroaromatic-containing group is not particularly limited, and is preferably in the range of 4 to 75, or 4 to 60, or 5 to 50, or 5 to 35, or 5 to 25, or 5 to 20.

As used herein, the term “halo” refers to an atom selected from fluoro, chloro, bromo, and iodo.

As used herein, the “bio-based content” is determined in accordance with ASTM-D6866 and is built on the same concepts as radiocarbon dating, but without use of the age equations. The analysis is performed by deriving a ratio of the amount of radiocarbon (¹⁴C) in an unknown sample to that of a modern reference standard. The ratio is reported as a percentage with the units “pMC” (percent modern carbon) with modern or present defined as 1950. If the material being analyzed is a mixture of present-day radiocarbon and fossil carbon (containing no radiocarbon), then the pMC value obtained correlates directly to the amount of biomass material present in the sample. Combining fossil carbon with present day carbon into a material will result in a dilution of the present day pMC content. By presuming 107.5 pMC represents present day biomass materials and 0 pMC represents petroleum derivatives, the measured pMC value for that material will reflect the proportions of the two component types. A material derived 100% from present day plant/tree would give a radiocarbon signature near 107.5 pMC. If that material was diluted with 50% petroleum derivatives, it would give a radiocarbon signature near 54 pMC. A bio-mass content result is derived by assigning 100% equal to 107.5 pMC and 0% equal to 0 pMC. In this regard, a sample measuring 99 pMC will give an equivalent bio-based content result of 93%.

According to an aspect of the present invention, a method for depolymerizing a polyurethane to provide a regenerated isocyanate compound in a single step is disclosed herein. The method includes contacting the polyurethane with a depolymerization agent in the presence of a solvent and a Lewis base. Any suitable depolymerization agent may be used, including, but not limited to a halide-containing boron compound, such as halo-organo borane compound or a boron trihalide. The regenerated isocyanate mixture may include a mixture of a regenerated isocyanate compound and at least one of an adduct of a polyol and the halide-containing boron compound, an adduct of a chain extender and the halide-containing boron compound, a residual amount of the Lewis base, or an acid-base adduct. In an embodiment, the regenerated isocyanate mixture may include a mixture of a regenerated isocyanate compound and at least two, or at least three, or all four of an adduct of a polyol and the halide-containing boron compound, an adduct of a chain extender and the halide-containing boron compound, a residual amount of the Lewis base, or an acid-base adduct.

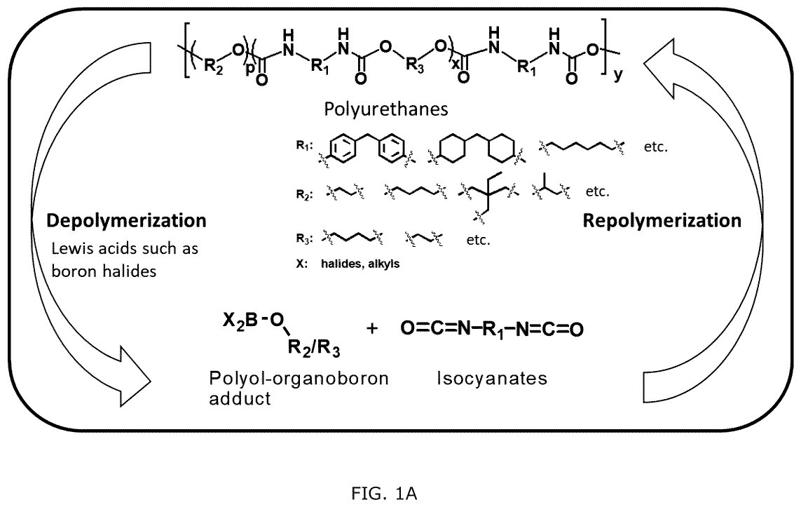

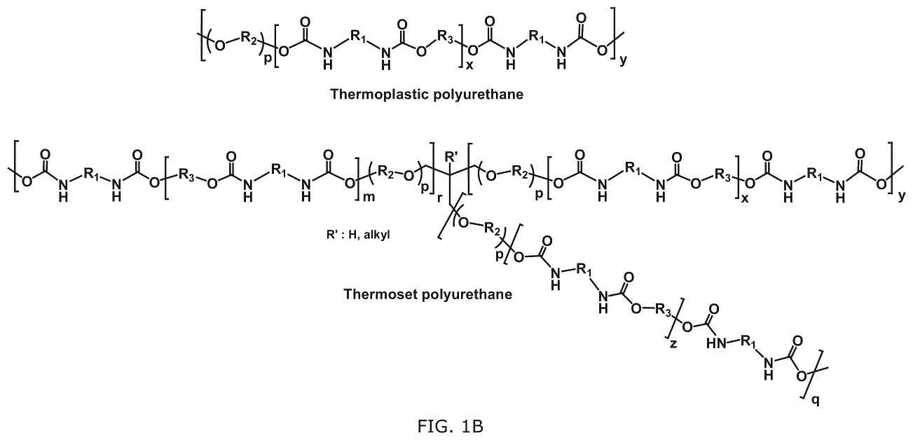

In an embodiment, the polyurethane is a thermoplastic polyurethane or a thermoset polyurethane, as represented by the structures shown in FIG. 1B. In the generalized structures of polyurethanes shown in FIG. 1B, R′ can be hydrogen or a C1-C10, or C1-C8, or C₁-C₅, or C₁-C₃ alkyl; each R₁ is independently (i) an unsubstituted or a substituted alkylene group having 2 to 75, or 2 to 50, or 2 to 25 or 2 to 15 or 2 to 10 carbon atoms where one or more carbon atoms in the alkylene group may be substituted by oxygen, nitrogen or sulfur atoms with the proviso that only carbon atoms are directly bonded to each of the two NH groups in the polyurethane backbone; or (ii) an unsubstituted or a substituted aromatic-containing group having 6 to 75, or 6 to 50, or 6 to 35, or 6 to 25, or 6 to 20 carbon atoms; or (iii) an unsubstituted or a substituted heteroaromatic-containing group having 4 to 75, or 3 to 60, or 5 to 50, or 5 to 35, or 5 to 25, or 5 to 20 carbon atoms; or (iv) an unsubstituted or a substituted cycloalkyl-containing group having 4 to 75, or 3 to 60, or 5 to 50, or 5 to 35, or 5 to 25, or 5 to 20 carbon atoms; or (v) an unsubstituted or a substituted heterocycloalkyl-containing group having 4 to 75, or 3 to 60, or 5 to 50, or 5 to 35, or 5 to 25, or 5 to 20 carbon atoms; or (vi) combinations thereof; and x, y, z, m, n, and r, each independently is 1 to 200, and p is 3 to 100. R₂ and R₃ can be independently selected from an alkylene group having at least 2 carbon atoms, where one of R₂ and R₃ can be an alkylene group having 2 to 10 carbon atoms, and the other of R₂ and R₃ can be an oligomeric or a polymeric segment having at least 6, or 10, or 25, or 35, or 50, or 60, or 75 carbon atoms; one or more carbon atoms is optionally substituted by oxygen, nitrogen, or sulfur atoms with the proviso that only carbon atoms are directly bonded to each of the end oxygen atoms in the polyurethane backbone.

In an embodiment, at least one of R₂ and R₃ contains a polyether, a polyester, a thioether, a polyether containing at least one thioether group, a polyimide, or combinations thereof.

In an embodiment, the amount of depolymerization agent is used in excess of the amount of urethane linkages present in the polyurethane, which is calculated as described hereinbelow. In another embodiment, the depolymerization agent is present in an amount of 0.1 to 10, or 0.2 to 6, or 0.3 to 3 molar equivalents with respect to the urethane linkages. As used herein, the molar amount of the urethane linkages was calculated from the molar amount of the isocyanate used in the synthesis of lab-made thermoplastic and thermoset PUs. It should be noted that as used herein, the molar amount of the urethane linkages is an estimated value.

According to an embodiment, the depolymerization agent can be a halide-containing boron compound such as a halo-organo borane compound or a boron trihalide. Any suitable halo-organo-borane compound can be used, including, but not limited to, β-chlorocatecholborane, β-bromocatecholborane, β-fluorocatecholborane, β-iodocatecholborane, or combinations thereof. Examples of boron trihalide include, boron trifluoride, boron trichloride, boron tribromide, and boron triiodide.

In another embodiment, the Lewis base may include one or more of triethylamine, pyridine, dimethylaminopyridine, triethylenediamine, 1,8-Diazabicyclo [5.4.0]undec-7-ene (DBU), 1,5-Diazabicyclo[4.3.0]non-5-ene (DBN), other amidine derivatives, diisopropylethylamine, other non-nucleophilic alkylamines, lithium diisopropylamide, and quinuclidine.

Any suitable solvent may be used. In an embodiment, the solvent is a non-polar solvent, including, but not limited to, toluene, hexane, heptane, benzene, cyclohexane, or combinations thereof.

According to various embodiments, the depolymerization can be carried out at a temperature in a range of 10° C. to 200° C., or 20° C. to 150° C., or 40° C. to 100° C. The depolymerization can be carried out for any suitable amount of time, for example for 5 min to 24 h, or 10 min to 12 h, or 15 min to 6 h, or 20 min to 5 hr. It should be noted that the time for carrying out the depolymerization is dependent upon the temperature at which the depolymerization is carried out.

In an embodiment, the step of depolymerizing is carried out in an inert environment such as, for example, nitrogen (N₂), argon, helium, or combinations thereof. In another embodiment, the depolymerizing is carried out in dry air, substantially free of moisture. In yet another embodiment, the depolymerizing is conducted under vacuum.

According to an embodiment of the present invention, the polyol comprises poly(tetramethylene oxide), polyethylene glycol, polyethylene glycol trimethylolpropane triether, polypropylene glycol, or combinations thereof. In another embodiment, the chain extender comprises 1,4-butanediol, glycerol, ethylene glycol, 1,6-hexanediol, or combinations thereof.

Suitable examples of an adduct of a polyol and a halo-organo borane compound, include, but are not limited to, catecholboron ethers. Suitable examples of an adduct of a chain extender and a halo-organo boron compound, include, but are not limited to, catecholboron ethers such as dicatecholboron ethyl ether. Suitable examples of an acid-base adduct, include, but are not limited to, triethylamine hydrochloride and dimethylaminopyridinium hydrochloride. A residual amount of the Lewis base may be present in a range of 1 to 30%, 1 to 10%, or 1 to 5% by mass, based on the total amount of Lewis base relative to the amount of depolymerizing agent. The adduct of a polyol and the halide-containing boron compound, the adduct of a chain extender and the halide-containing boron compound or the acid-base adduct may be present in an amount in a range of 5 to 90%, or 10 to 85%, or 15 to 75% by mass, based on the total amount of reaction mixture.

In an aspect of the invention, the method may further include a step of isolating recovered isocyanate from the regenerated isocyanate mixture by treatment of the regenerated isocyanate mixture with at least one of chromatography, distillation (e.g., as vacuum distillation), differential extraction, or crystallization. In an embodiment, the step of isolating recovered isocyanate compound from the regenerated isocyanate mixture is carried out after the step of depolymerizing the polyurethane.

Another aspect of the present invention is a method of forming a polyurethane comprising contacting the isolated recovered isocyanate from the method as disclosed hereinabove, with one or more polyols and/or chain extenders in the presence of a polymerization catalyst, and, an optional solvent.

In an embodiment, the step of contacting the isolated recovered isocyanate further comprises contacting the isolated recovered isocyanate first with the polymerization catalyst and a polyol at a first temperature in the range of 20° C. to 120° C., or 20° C. to 90° C., or 60° C. to 70° C. for a first amount of time in a range of 5 min to 12 h, or 10 min to 5h, or 15 min to 1 hr, to form a pre-polymer. The pre-polymer is then chain extended to form a high molecular weight polymer by adding one or more chain extenders to the reaction mixture and stirring at a second temperature in the range of 20° C. to 120° C., or 50° C. to 120° C., or 90° C. to 110° C. for a second amount of time in a range of 10 min to 48 h, or 15 min to 24 h, or 30 min to 16 h, or 1 h to 6 h. In an embodiment, the polymerization catalyst and a polyol are dissolved in the same solvent as isolated recovered isocyanate.

Any suitable polymerization catalyst may be used, such as, a tin-containing catalyst or an amine catalyst. Suitable examples of the tin-containing catalyst may include dibutyltin dilaurate, dioctyltin dilaurate, dicotyl carboxylate, stannous octoate, or mixtures thereof. Any suitable amine catalyst may be used, including but not limited to, 1,4-diazabicyclo[2.2.2]octane (DABCO), dimethylethanolamine, triethylenediamine, or mixtures thereof.

In an embodiment, the optional solvent is the same as the solvent used in the step of depolymerizing the polyurethane. And in another embodiment, the optional solvent is different from that used in the step of depolymerizing the polyurethane. Suitable examples of the optional solvent include, but are not limited to, N,N-dimethylformamide, N,N-dimethylacetamide, or tetrahydrofuran.

In another aspect of the present invention, a composition may include the polyurethane prepared according to the method as disclosed hereinabove and a halide-containing boron compound. The composition may include any suitable amount of polyurethane, such as in an amount of at least 1%, or 2%, or 5%, or 10%, or 25%, or 50%, or 75%, 90%, or 95% by mass, based on the total mass of the composition. In an embodiment, the halide-containing borane compound may be present in the composition in an amount of at least 0.1 ppm, or 0.10 ppm, or 10 ppm, or 100 ppm, or 1000 ppm, as detected by energy-dispersive X-ray spectroscopy (EDX) or inductively coupled plasma optical emission spectroscopy (ICP-OES). In another embodiment, the composition may further include trace amount of at least one of an adduct of a polyol and the halide-containing boron compound, an adduct of a chain extender and the halide-containing boron compound, a residual amount of the Lewis base, or an acid-base adduct, as disclosed hereinabove. As used herein, the trace amount refers to less than 1000 ppm, or less than 500 ppm, or less than 100 ppm, or less than 50 ppm, or less than 10 ppm, or less than 1 ppm, or less than 0.5 ppm, or less than 0.1 ppm.

In an aspect of the invention, the composition may further include one or more of a virgin polyurethane, an inert filler, and an additive. The virgin polyurethane may be present in any suitable amount, such as from 0 to 90%, or 0% to 50%, or 0% to 25%, based on the total amount of the composition.

Suitable examples of inert fillers include, but are not limited to, biomass, lignin, cellulose, natural or synthetic fibers, silica, or recycled polymers. The inert fillers may be present in any suitable amount, such as from 0 to 80%, or 1% to 50%, or 1% to 25%, based on the total amount of the composition.

Suitable examples of additives include, but are not limited to, antioxidants, processing aids, lubricants, plasticizers, tackifiers, flame retardants, dyes, or stabilizers. The additives may be present in any suitable amount, such as from 0 to 60%, or 0% to 20%, or 0% to 5%, based on the total amount of the composition.

In an embodiment, the composition has a bio-based content in the range of 20 to 80% according to ASTM-D6866.

In an aspect of the present invention, an article includes the composition as disclosed herein.

In another aspect of the present invention, there is an integrated method comprising the steps of providing a regenerated isocyanate mixture obtained by reacting a polyurethane with a depolymerization agent in the presence of a solvent and a Lewis base, purifying the regenerated isocyanate mixture by at least one of chromatography, distillation (e.g., as vacuum distillation), differential extraction, or crystallization to obtain an isolated recovered isocyanate compound; and forming a recycled polyurethane by reacting the recovered isocyanate with one or more polyols in the presence of a catalyst, wherein the one or more polyols comprises a recovered polyol, a bio-derived polyol, a petroleum-based polyol, or a mixture thereof.

### Model Diurethane Deconstruction

The objective of the present invention is to recover isocyanates directly from PUs, and a depolymerization agent that could efficiently transform the urethane/carbamate linkages in the polymer to isocyanate groups in a single step.

Previous literature reported the use of catecholborane halides and boron halides for the deconstruction of small molecule carbamates/diurethanes with near-quantitative conversion. As a proof-of-concept, an aromatic diurethane (DU1) was synthesized from MDI and methanol (not shown) and deconstructed the compound using β-chlorocatecholborane in the presence of triethylamine with toluene as a solvent at 80° C. and ambient pressure. The successful deconstruction of DU1 and the reformation of MDI were confirmed by ATR-FTIR and NMR spectroscopy (not shown). The ATR-FTIR spectrum showed the appearance of a peak at ˜2270 cm-1 that corresponded to —N═C═O stretching (not shown). Additionally, the characteristic urethane peaks from —C═O stretching, —NH stretching, and —NH bending at ˜1730-1690, 3300, and 1500 cm⁻¹, respectively, disappeared. Additional experiments using β-chlorocatecholborane without a Lewis base (i.e., triethylamine) and vice versa were conducted as controls, and further details can be found in the Comparative Example Nos. 1 and 2 disclosed hereinbelow.

### Thermoplastic PU Depolymerization

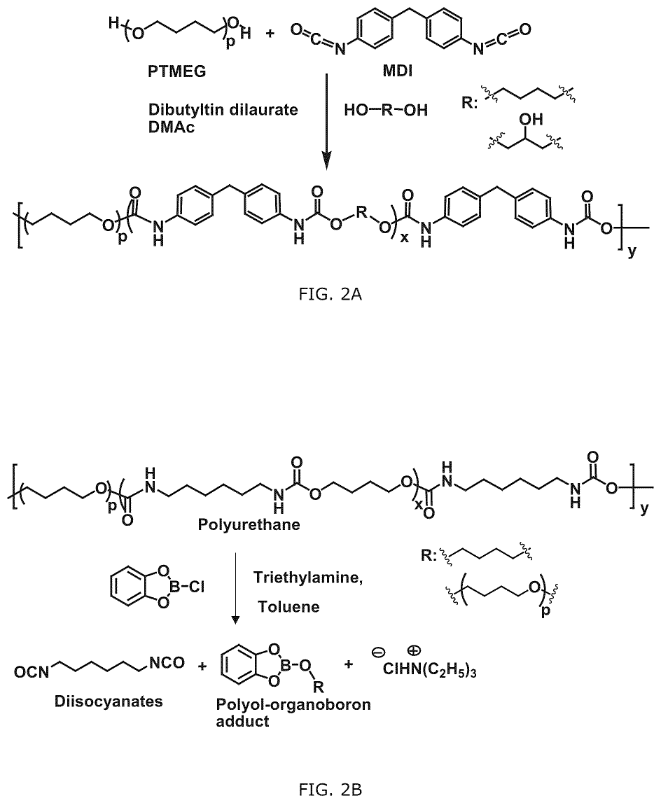

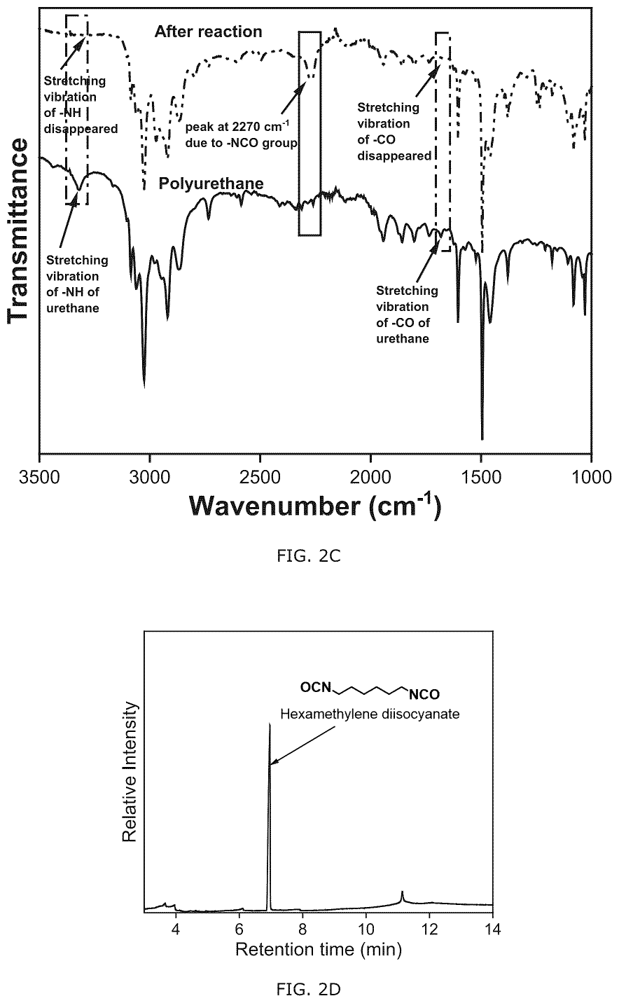

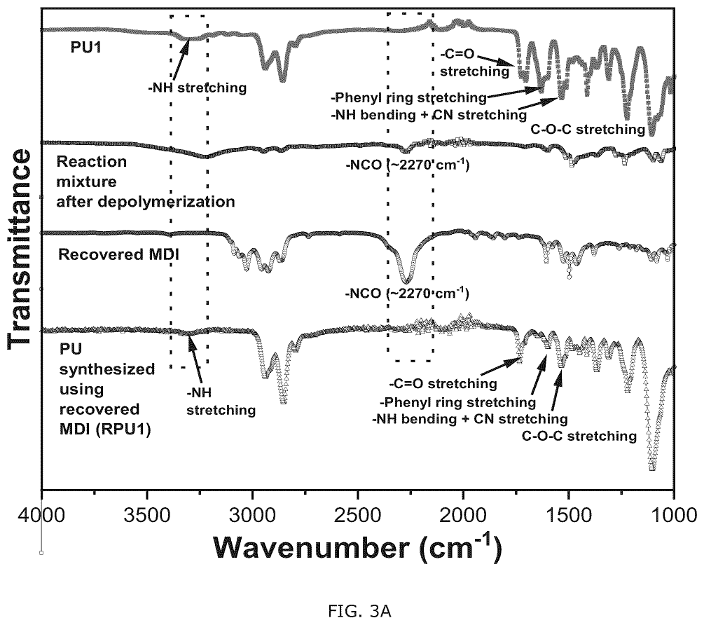

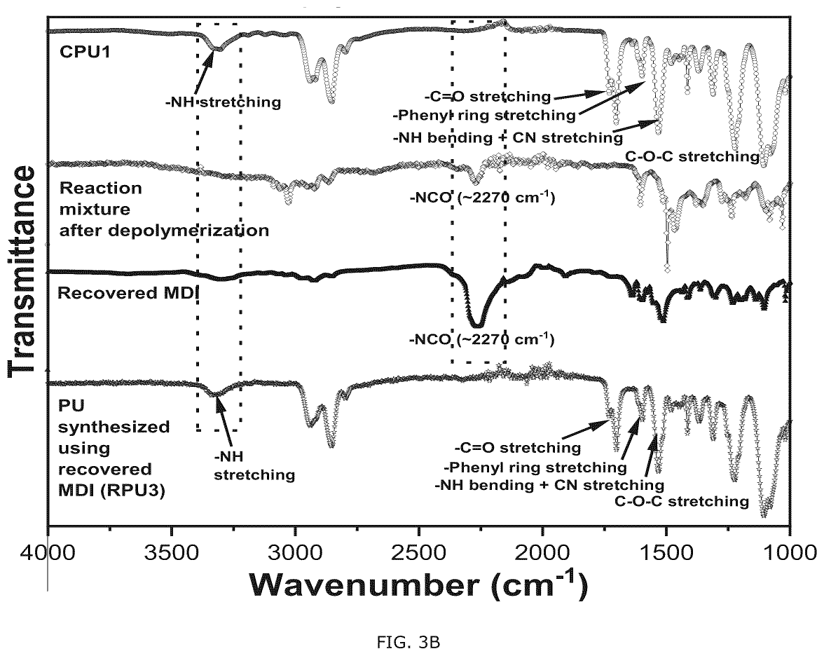

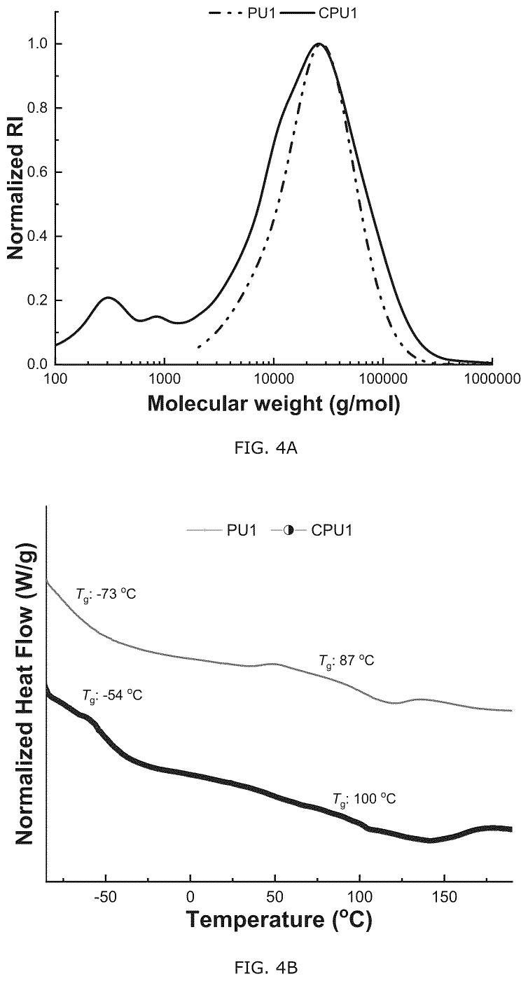

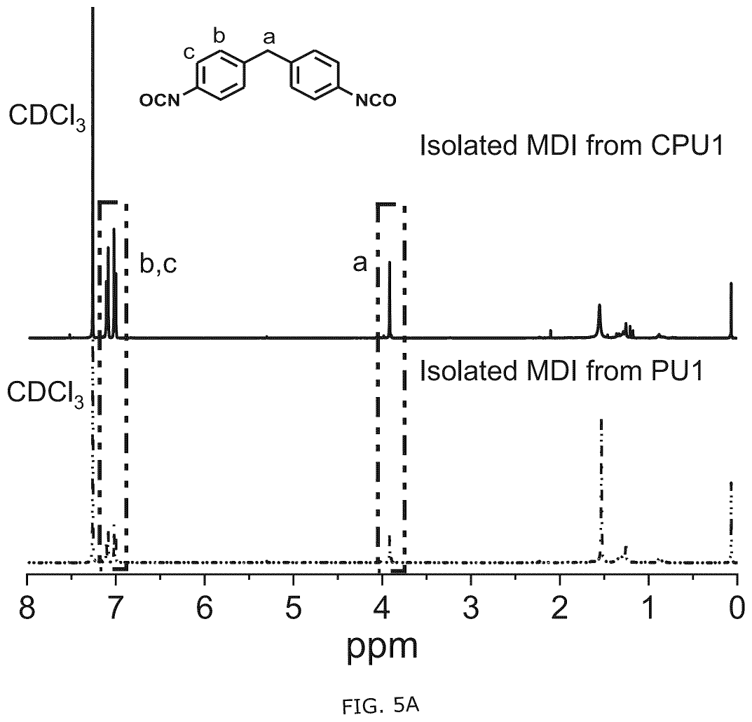

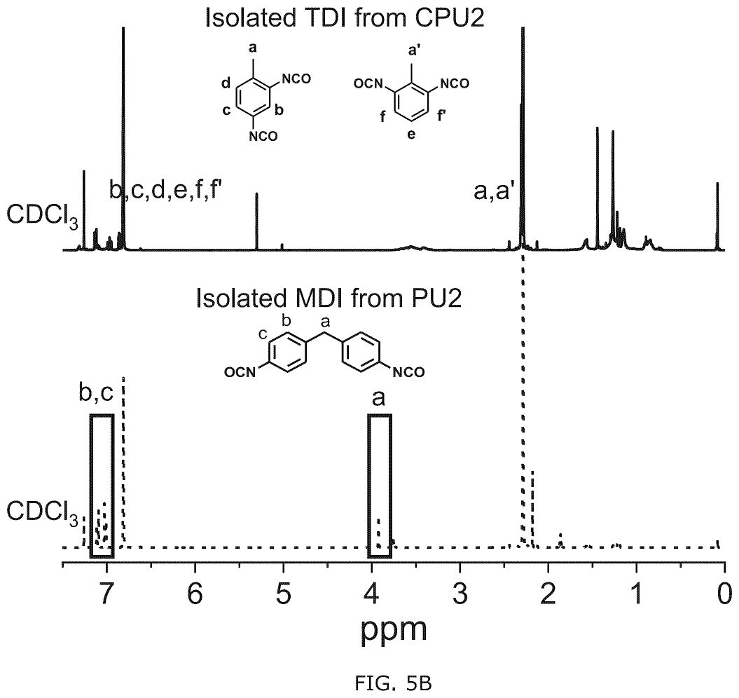

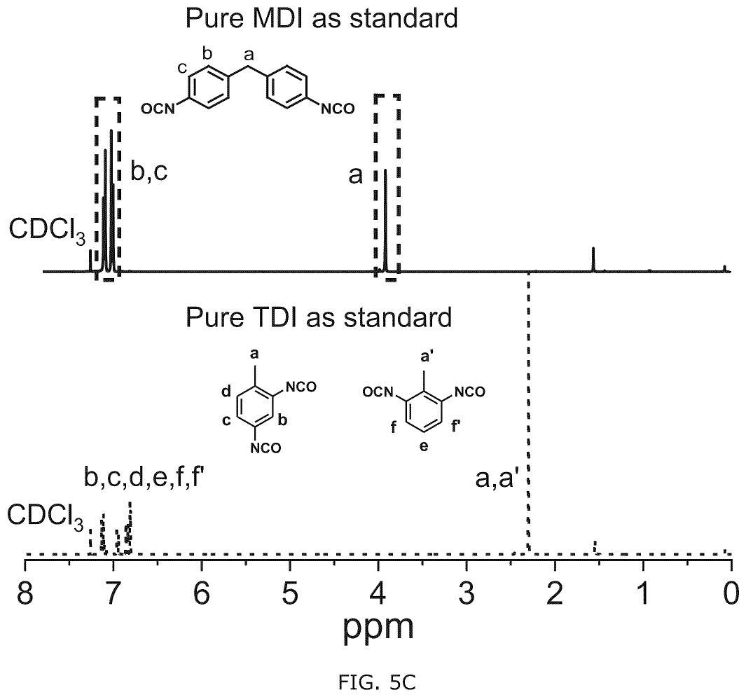

Two thermoplastic PUs (PU1 (lab synthesized) and CPU1 (commercially available)) were depolymerized using the same methodology as used for the model diurethane deconstruction, as disclosed hereinbelow in Example No. 1. The PU1 was synthesized using MDI as the hard segment, poly(tetramethylene oxide) (PTMO) as the soft segment, and 1,4-butanediol (BDO) as the chain extender (FIG. 2A; Table 1), and CPU1, an MDI-based PU, was purchased from a commercial supplier and stripped of additives by dissolution and precipitation prior to use. The successful synthesis of PU1 was confirmed by ATR-FTIR spectroscopy as shown in FIG. 3A. The disappearance of the isocyanate peak at ˜2270 cm⁻¹ and the presence of urethane carbonyl stretch (˜1730-1690 cm⁻¹), urethane-NH stretch (˜3300 cm⁻¹), and urethane-NH bend (˜1500 cm⁻¹) peaks confirmed the formation of the PU. Similarly, CPU1 was characterized by ATR-FTIR spectroscopy and exhibited the same characteristic urethane peaks (FIG. 3B). The molecular weights of both thermoplastic PUs were determined by gel permeation chromatography (GPC) analysis (FIG. 4A and Table 1), and differential scanning calorimetry (DSC) was conducted to assess the thermal characteristics (e.g., glass transition temperature [T_{g}] and crystallization/melting behavior) of the materials as shown in FIG. 4B.

TABLE 1
Compositional details for the synthesized Polyurethanes

| | | Polymer Sample | | | |
|---|---|---|---|---|---|
| | | PU1 | CPU1 | PU2 | CPU2 |
| Isocyanate | | MDI | MDI | MDI | TDI |
| Polyol | | PTMO | PTMO | PTMO | Unknown |
| Chain | | BDO | BDO | Glycerol | Unknown |
| Type | | Thermoplastic | Thermoplastic | Thermoset | Thermoset |
| M_{w} | (g/mol)^{a} | 30,700 | 32,600 | — | — |
| Mₙ | (g/mol)^{a} | 14,700 | 11,200 | — | — |
| Dispersity^{a} | (M_{w}/Mₙ) | 1.7 | 2.9 | — | — |
| PU | conv. (%)^{b} | 66.1 | — | 63.2 | — |

^{a}Calculated by Gel Permeation Chromatography using PMMA standards.
^{b}PU conversion (%) was determined gravimetrically.

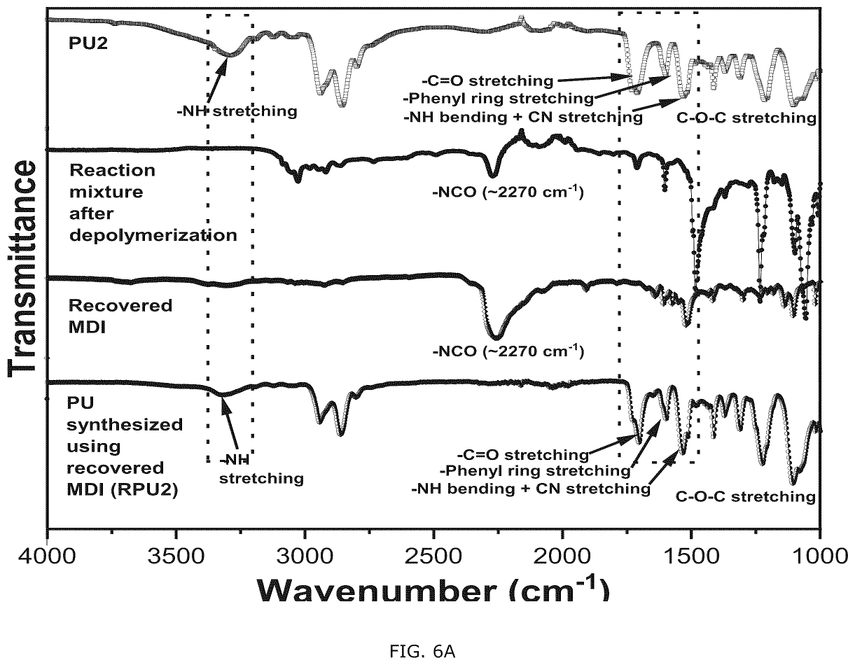

The synthesized thermoplastic polyurethane (PU1) and commercially sourced thermoplastic polyurethanes (CPU1) were successfully depolymerized using β-chlorocatecholborane in presence of triethylamine at 80° C. in toluene, as illustrated in the scheme shown in FIG. 2B. The formation of MDI was confirmed by ATR-FTIR spectroscopy, and the appearance of the —N═C═O stretching peak at ˜2270 cm⁻¹ and the disappearance of the characteristic urethane peaks from —C═O stretching, —NH stretching, and —NH bending were consistent in both cases (FIG. 3A and FIG. 3B). The regenerated isocyanate compounds were separated by column chromatography to obtain white solids that were characterized by ATR-FTIR and 1H NMR spectroscopy. The ATR-FTIR spectra both showed a peak at ˜2270 cm⁻¹ corresponding to —N═C═O stretching (FIGS. 3A and 3B), confirming the presence of isocyanates. FIG. 5A shows the 1H NMR spectra (in CDCl₃) of the white solids with a spectrum for a pure MDI standard for reference. The isolated MDI yield for PU1 was 23%. For CPU1, because the initial content of MDI in the commercially sourced thermoplastic polyurethane was unknown, it was difficult to report the isolated yield. About 58 mg of MDI from 500 mg of CPU1 material was obtained after column chromatography (yield of 12 wt. %, based on the amount of the polyurethane (CPU1)).

The depolymerization reactions of PU1 and CPU1 both generated MDI; however, the product characterization for CPU1 was significantly more challenging than for PU1 because the composition of the commercially sourced sample was unknown. For instance, the ATR-FTIR spectrum for depolymerized CPU1 exhibited some of the characteristic urethane peaks, indicating incomplete conversion to isocyanate. Additionally, excess β-chlorocatecholborane and triethylamine were used for the depolymerizations of PU1 and CPU1, but the unknown composition and the presence of residual additives (e.g., antioxidants, processing aids, etc.) in CPU1 could have interfered with the depolymerization or reacted with isocyanates that were formed to generate byproducts. Yields also cannot be compared directly between PU1 and CPU1 as the MDI contents are not the same and are unknown in the case of CPU1. Additionally, an aliphatic isocyanate-based thermoplastic PU, synthesized using hexamethylene diisocyanate (HDI) as the hard segment, PTMO as the soft segment, and BDO as the chain extender, was successfully depolymerized using β-chlorocatecholborane in presence of triethylamine at 80° C. in toluene, as illustrated in the scheme shown in FIG. 2B. The formation of HDI was confirmed by ATR-FTIR spectroscopy, and the appearance of the —N═C═O stretching peak at ˜2270 cm⁻¹ and the disappearance of the characteristic urethane peaks from —C═O stretching, —NH stretching, and —NH bending were consistent with prior observations (FIG. 2C). Also, the presence of HDI in the product mixture was confirmed by gas chromatography-mass spectrometry (GC-MS) (FIG. 2D).

### Thermoset PU Depolymerization

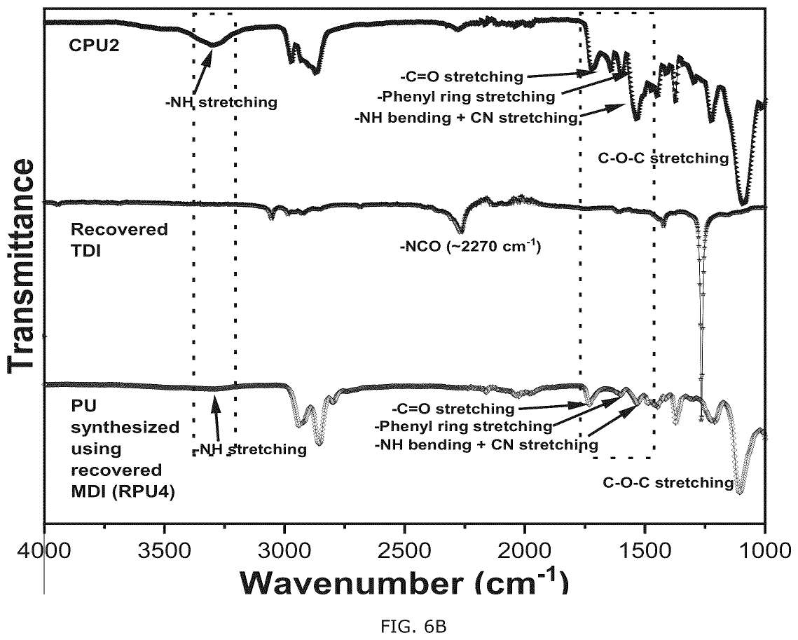

Thermoset PUs are difficult to recycle by any of the common recycling techniques, but the β-chlorocatecholborane-based depolymerization technique has been demonstrated to apply to both a laboratory-synthesized (PU2) and a commercially-sourced (CPU2) thermoset polyurethane. The same depolymerization conditions were applied to both thermosets, and the formation of the corresponding isocyanates was confirmed by ATR-FTIR spectroscopy (FIGS. 6A-6B). The ATR-FTIR spectra indicated the appearance of the characteristic —N═C═O stretching peak along with the disappearance of the urethane —C═O stretching, —NH stretching, and —NH bending peaks. The isocyanates were separated by column chromatography. MDI was recovered at 20% yield (isolated recovered isocyanate yield) from PU2 and characterized by ATR-FTIR and 1H NMR spectroscopy (FIGS. 6A and 5C, respectively). For CPU2, TDI similarly was separated by column chromatography and isolated with a yield of <5 wt. %, based on the total amount of the polymer. TDI also was characterized by ATR-FTIR and ¹H NMR spectroscopy. The ATR-FTIR spectrum showed the —N═C═O stretching peak (FIG. 6B), and the NMR spectrum was comparable to that of an authentic standard (FIG. 5C).

Thermoset PUs historically are difficult to recycle, and the application of this depolymerization technique could play a major role in improving the circularity of thermoset PUs. However, the isocyanate yields from the two thermoset PUs (PU2 and CPU2) were somewhat lower than the isocyanate yields from the thermoplastic PUS (PU1 and CPU1) at 20 wt. % and <5 wt. % vs. 23 wt. % and 12 wt. %, respectively. This discrepancy may arise from the inability of the depolymerization agent to access the thermoset materials and choosing solvents that better swell the thermoset PUs or grinding the thermosets more finely to increase surface area could result in higher conversions. The presence of additives in CPU2 also may have negatively impacted conversion similar to the thermoplastic case. Additionally, the isolation of TDI from CPU2 was significantly easier than the isolation of MDI from other PUs because of the comparatively high solubility of TDI in most organic solvents (e.g., toluene, ethyl acetate, and hexanes) that are commonly used for column chromatography. Thus, the development of improved separations techniques to isolate isocyanates from these depolymerization mixtures may significantly improve yields.

### PUs Synthesized Using Recovered MDI/TDI

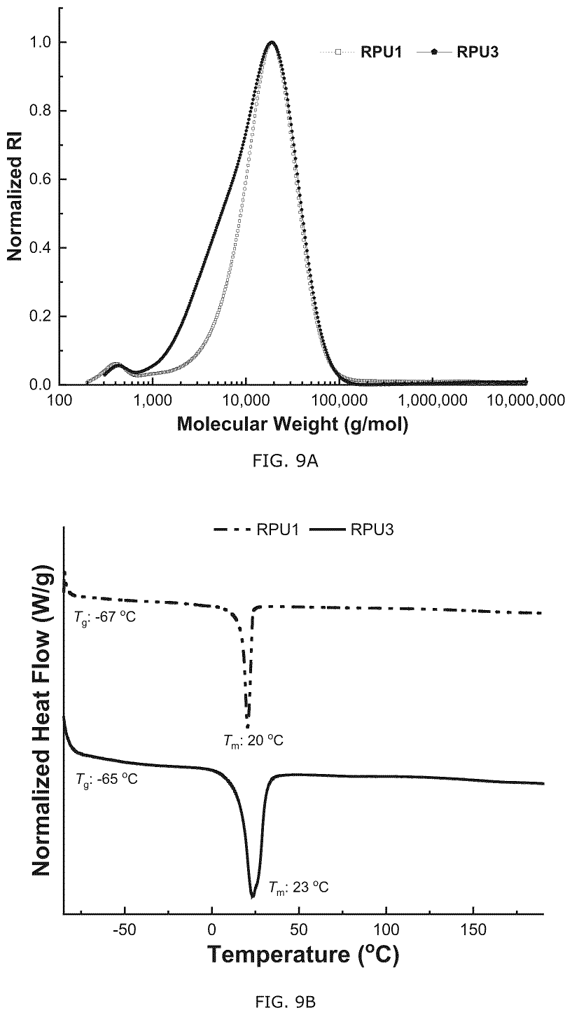

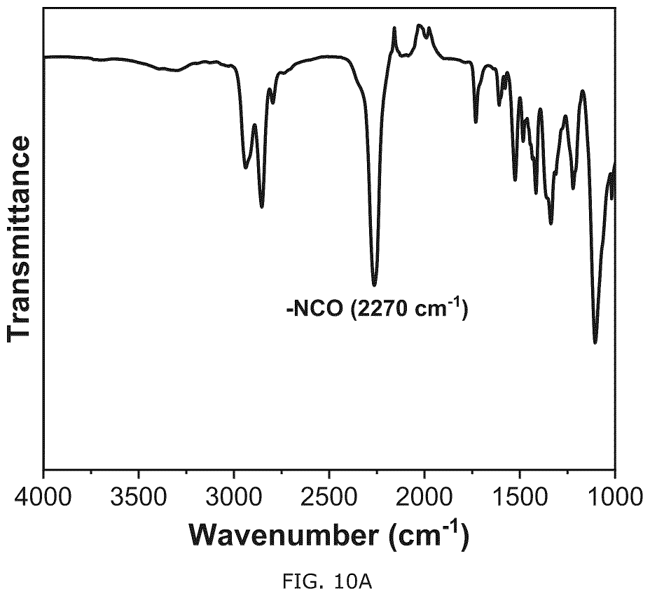

Recovered isocyanates from PU1, CPU1, PU2, and CPU2 were used to regenerate new PU materials (RPU1-RPU4; see Table 2) with the same composition as PU1 (PTMO macrodiol and BDO chain extender). The conversion of the isocyanates and —OH functional groups to urethane linkages was confirmed by ATR-FTIR spectroscopy for each system as shown in FIGS. 3A, 4B, 6A, and 6B, respectively. In all cases, the —N═C═O stretching peak disappeared, and characteristic urethane peaks reappeared. GPC was used to measure the molecular weight distributions of the resynthesized PUs, and weight average molecular weight (M_{w}) values ranged from 20,800 g/mol to 6,900 g/mol with dispersity (Ð) varying from 1.7 to 2.2. The thermal characteristics of the resynthesized PUs also were assessed by DSC (Table 2). RPU1 and RPU3 showed similar thermal transitions for the soft segment T_{g}, and melting temperature (Tₘ) at around ˜−65° C. and 20° C. with a slight shift for each transition. Furthermore, neither RPU1 nor RPU3 exhibit the hard segment T_{g} at higher temperatures. However, the thermal transition of RPU2 is different from that of RPU1 and RPU3. RPU2 exhibited a T_{g} of −65° C., crystallization temperature (T_{c}) of −25° C., a melting point, (Tₘ,) of 18° C. for the soft segment, and two T_{g}s at 103° C. and 148° C. for hard segment. However, the reasons for the lack of a transition and/or additional transitions are unclear (FIGS. 9B and 8).

TABLE 2
Compositional details and key characteristics for the resynthesized PUs.

| Sample | | PU1 | RPU1 | RPU2 | RPU3 | RPU4 |
|---|---|---|---|---|---|---|
| Isolated Isocyanate | | — | PU1 | PU2 | CPU1 | CPU2 |
| recovered from | | | | | | |
| Isocyanate | | MDI | MDI | MDI | MDI | TDI |
| Polyol | | PTMO | PTMO | PTMO | PTMO | PTMO |
| Chain extender | | BDO | BDO | BDO | BDO | BDO |
| M_{w} | (g/mol) | 30,700 | 20,800 | 6,900 | 17,700 | 9,600 |
| Mₙ | (g/mol) | 14,700 | 12,000 | 3,900 | 8,100 | 5,800 |
| Ð | (M_{w}/Mₙ) | 1.7 | 1.7 | 1.8 | 2.2 | 1.7 |

It should be noted from Table 2, that thermoplastic polyurethanes were resynthesized using the isolated recovered isocyanates; however, for RPU3, from commercial PU, the measured weight average molecular weight were significantly lower than the molecular weight of the starting PU (17,700 g/mol (RPU3) vs 32,600 g/mol (CPU1)). This discrepancy could be explained by impurities in the recovered isocyanates leading to mismatched stoichiometry, and more careful purification of the isocyanates may enable better control over molecular weight. Additionally, further recycling of resynthesized PUs was not explored, but given the equivalence in molecular weight and thermal characteristics, they are expected to behave similar to virgin PUs with the same compositions.

### CONCLUSION

A ‘one-step’ technique for the depolymerization and regeneration of thermoplastic and thermoset polyurethane (PU) materials has been reported for the first time and applied to both lab-made and commercially sourced PUs. The method uses an inexpensive halide-containing boron compounds as the depolymerization agent and directly produces isocyanates under mild reaction conditions (<100° C. and ambient pressure). The isocyanates were isolated with up to 23 wt. % yield, based on either starting isocyanate content or the amount of the starting polymer and used to regenerate new PU materials with molecular weights and thermomechanical properties that were equivalent to virgin PUs. Overall, this novel recycling method enables circularity for conventional PUs and could minimize the need for the production of new isocyanates.

Within this specification, embodiments have been described in a way which enables a clear and concise specification to be written, but it is intended and will be appreciated that embodiments may be variously combined or separated without departing from the invention. For example, it will be appreciated that all preferred features described herein are applicable to all aspects of the invention described herein.

### EXAMPLES

Examples of the present invention will now be described. The technical scope of the present invention is not limited to the examples described below.

### Materials

All solvents used were purchased from Sigma-Aldrich. All dry solvents were purchased from Sigma-Aldrich. Unless specified, all chemicals used were purchased from Sigma-Aldrich. Dibutyltin dilaurate (DBTDL) was stored under dried nitrogen and used as received. Anhydrous N,N-dimethylacetamide (DMAc), anhydrous methanol, anhydrous dichloromethane, and anhydrous toluene were used as received. Poly(tetramethylene oxide) (PTMO; Mₙ: 2,000 g/mol) was dehydrated and degassed under vacuum at 80° C. for 24 h and stored under a dry nitrogen (N₂) atmosphere prior to use. 1,4-butanediol (BDO) was vacuum distilled and stored under dried nitrogen prior to use. Methylene diphenyl diisocyanate (MDI), purchased from TCI America, was vacuum distilled and stored under dried N₂ prior to use. β-Chlorocatecholborane was purchased from TCI and used as received. Triethylamine was used as received. Boron trichloride (ca. 13% in Toluene, ca. 1.0 mol/L) were purchased from TCI and used as received. CPU1, an MDI-based PU, was obtained from a commercial supplier (Lubrizol) and stripped of additives by dissolution and precipitation prior to use.

### Characterization

### Attenuated Total Reflectance Fourier-Transform Infrared (ATR-FT-IR)

ATR FT-IR spectra were recorded on a Thermo Nicolet NEXUS 670 FTIR spectrometer using a mercuric cadmium telluride (MCT) detector.

### Gel Permeation Chromatography (GPC)

The number-average molecular weight (Mₙ), weight average molecular weight (M_{w}) and molecular weight distributions (dispersity) of soluble polymers were determined in DMAc using a TOSOH EcoSEC Elite GPC instrument (HLC-8420) equipped with a refractive index (RI) detector. The DMAc mobile phase contained 0.5 wt. % LiBr. A series of 8 nearly monodisperse poly(methyl methacrylate) (PMMA) standards (Agilent Technologies) with molecular weight values ranging from 1,840 to 2,210,000 g/mol were used to calibrate the instrument.

### NMR Spectroscopy

¹H NMR spectra were obtained either on a Bruker AV 400 MHz or AVIII 600 MHz spectrometer.

### Thermal Analysis (DSC)

Thermal analysis of the polyurethanes was performed using a TA Instruments Discovery Series DSC instrument. Three heating and two cooling cycles were performed between −90° C. and 200° C. under a N₂ atmosphere at a heating/cooling rate of 20° C. min⁻¹. The data from the second heating cycle are presented.

### Methods

### Calculated Molar Amount of Urethane Linkages in a Polyurethane:

Molar amount of urethane linkages was calculated as follows:

Weight of lab-made thermoplastic polyurethane PU1 taken for depolymerization: 500 mg

Calculated amount (in mg) of isocyanates (MDI) in 500 mg of PU1: 153 mg

Mmols of isocyanate (MDI) in 500 mg of PU1: 0.611 mmol

Considering, one molecule of MDI has two isocyanate functional group (—NCO), mmols of urethane linkages=2×mmols of isocyanate (MDI) in 500 mg of PU1=2×0.611 mmol=1.222 mmols

β-chlorocatecholborane was used in excess of urethane linkages, such as molar ratio of Urethane linkages: β-chlorocatecholborane of 1:1.25.

Hence, for 1.222 mmols of urethane, 1.5275 mmols of β-chlorocatecholborane was used (1.222*1.25==1.5275 mmols).

### Reference Example 1: Synthesis of Model Diurethane DU1

4,4′-methylene phenylene diisocyanate (MDI) (4.8 mmol) was added to excess of anhydrous, dry methanol (5 mL) while stirring at room temperature. Formation of a white precipitate was observed, and the reaction was stirred at 65° C. for 18 h. Thereafter, the excess organic solvent was concentrated under reduced pressure. The product was isolated and purified by column chromatography. 1H NMR (600 MHZ, DMSO-d6, δ ppm): 9.51 (s, 2H, —NHCOO), 7.34 (d, 4H, ArH), 7.09 (d, 4H, ArH), 3.79 (s, 2H, ArCH2Ar), 3.64 (s, 6H, —OCH3).

### Reference Example 2: General Procedure for the Urethane/Carbamate to Isocyanate Transformation Using Model Carbamate DU1 and β-Chlorocatecholborane

DU1 (200 mg) and dry toluene (2 mL) were added to a round-bottom flask. Separately, triethylamine (0.21 mL) and β-chlorocatecholborane (235 mg) were dissolved in 2 mL of dry toluene, and the solution was added dropwise to the polymer solution. The resulting mixture was heated at 80° C. for 2 h. After the reaction, an aliquot from the reaction mixture was analysed by ATR-FTIR spectroscopy using a highly sensitive MCT detector.

### Reference Example 3: General Procedure for the Synthesis of Polyurethane PU1

To minimize side reactions with water, all of the polymerizations were carried out inside a glovebox. In a typical reaction, an excess amount of MDI (2.58 g, 15.3 mmol) was taken in a round-bottomed flask fitted with an additional funnel, and 5 mL of DMAc was added to it. PTMO (5.12 g, 5.1 mmol) and five drops of DBTDL were dissolved in another 25 mL of DMAc and added to the addition funnel. The PTMO/catalyst solution was slowly (over a period of 1 h) added to the MDI solution. The reaction mixture then was stirred at 65° C. for 3 h to form the pre-polymer. The pre-polymer was then chain extended to form a high molecular weight polymer by adding the chain extender BDO (0.923 g, 10.2 mmol) to the reaction mixture and stirring at 85° C. for 24 h. The reaction mixture was precipitated into a large excess of warm distilled water and the resulting polyurethane was washed with methanol and dried under dynamic vacuum until constant weight.

### Example 1: Depolymerization of Thermoplastic Polyurethane

Thermoplastic polyurethane PU1 (500 mg) was added a round-bottom flask with 10 mL of dry toluene. The mixture was stirred at 20° C. for 12 h. A solution of triethylamine (0.21 mL) and β-chlorocatecholborane (235 mg) in 5 mL of toluene was added dropwise to the polymer solution, and the resulting mixture was refluxed at 80° C. for 2 h. After the reaction, an aliquot from the reaction mixture was analyzed by ATR-FTIR spectroscopy using a highly sensitive MCT detector. The regenerated isocyanate compound was separated by column chromatography in a 23 wt. % yield (isolated recovered isocyanate compound yield).

Depolymerization procedure similar to that used for PU1, was used for commercially purchased thermoplastic polyurethane (CPU1), which was stripped of additives by dissolution and precipitation prior to use. About 58 mg of isolated recovered MDI was obtained from 500 mg of CPU1 material.

### Example 2: Depolymerization of Thermoset Polyurethane Using β-Chlorocatecholborane

The depolymerization procedure, similar to that used for PU1, was used for a synthesized thermoset polyurethane (PU2) and a commercially purchased thermoset polyurethane (CPU2). PU2 was synthesized using MDI, PTMO, and glycerol, and CPU2 was obtained from a kitchen sponge that was manufactured using TDI. The CPU2 sample was washed with tetrahydrofuran three times to remove any organic-soluble additives and/or dyes and dried under dynamic vacuum at 40° C. prior to depolymerization. The same depolymerization conditions (excess β-chlorocatecholborane, triethylamine, and toluene at 80° C.) were applied to both thermosets, and the formation of the corresponding isocyanates was confirmed by ATR-FTIR spectroscopy (FIG. 4) About 30 mg of isolated recovered MDI was obtained from 500 mg of PU2 material. About 10 mg of isolated recovered MDI was obtained from 500 mg of CPU2 material.

### Example 3: Effect of Temperature on the Product Yield

Polyurethane CPU1 (500 mg) was added to a round-bottom flask with 10 mL of dry toluene. A solution of triethylamine (0.21 mL) and β-chlorocatecholborane (235 mg) in 5 mL of dry toluene was added dropwise to the polymer solution, and the resulting mixture was stirred at a certain temperature (0, 50, 80, or 100° C.) for 2 h, as summarized in Table 3. After each reaction was finished, aliquots were analysed by ATR-FTIR spectroscopy using a highly sensitive MCT detector. The regenerated isocyanate compound was separated by column chromatography with yields ranging from 6.8-12 wt. %, based on the amount of the polyurethane (CPU1) (isolated yield).

TABLE 3
Yield as a function of temperature

| | | | Wt. %, based on the |
|---|---|---|---|
| | | Isolated recovered | amount of the |
| Entry | Temperature | isocyanate Yield^{a} | polyurethane (CPU1) |
| 1 | 0° C. | No depolymerization | — |
| 2 | 50° C. | 34 mg | 6.8 |
| 3 | 80° C. | 58 mg | 12 |
| 4 | 100° C. | 46 mg | 9.1 |

^{a}Purification using column chromatography

### Example 4: Effect of Catalyst

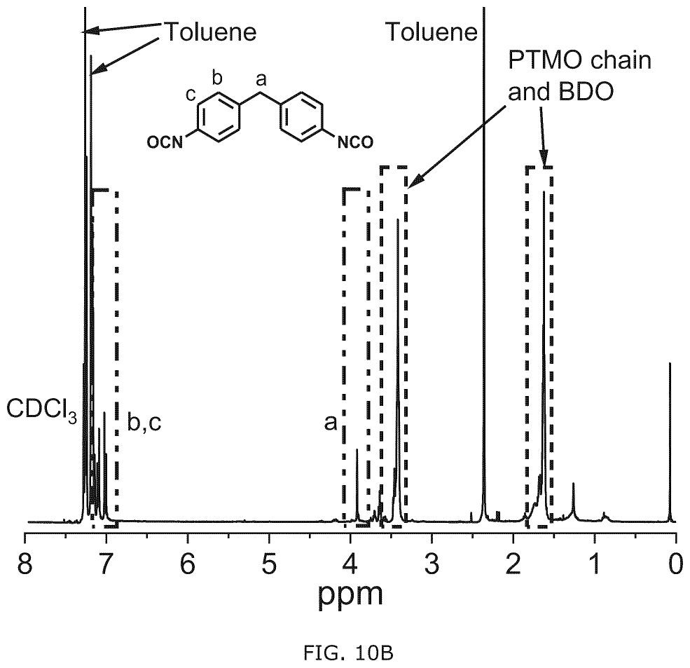

The depolymerization procedure, similar to that used for PU1 (MDI based polyurethane with PTMO and BDO), in Example No. 1, was used except that boron trichloride was used instead of β-chlorocatecholborane. The amounts of CPU1 and triethylamine were kept same as in Example No. 1. 444 μL of boron trichloride in toluene was used for the reaction. The temperature and reaction time were also kept same as in Example No. 1. After the reaction, an aliquot from the depolymerization reaction mixture was analysed by ATR-FTIR spectroscopy and characteristic peak of the isocyanate at 2270 cm⁻¹ was observed (FIG. 10A). Characterization of the depolymerization reaction mixture by NMR spectroscopy showed signals from isocyanates, polyol, and chain extender (FIG. 10B).

### Example 5: General Procedure for the Synthesis of Polyurethane (RPU1-RPU4) Using Recovered Isocyanates from PU1, PU2, CPU1, and CPU2

Polyurethanes were synthesized using the using recovered isocyanates from PU1, PU2, CPU1, and CPU2 obtained in Example Nos. 1 and 2 following the procedure described in the Reference Example 2. However, after the PTMO/catalyst solution was slowly added into the MDI solution the reaction was stirred at 65° C. for 25 min to form the pre-polymer. The pre-polymer was then chain extended to form a high molecular weight polymer by adding BDO to the reaction mixture and stirring at 100° C. for 34 h. The reaction mixture was precipitated into a large excess of cold methanol and dried under dynamic vacuum until constant weight. The experimental details are summarized in Table 4 below.

TABLE 4
Repolymerization of recovered isocyanates

| | Sample | | | |
|---|---|---|---|---|
| | RPU1 | RPU2 | RPU3 | RPU4 |
| Isocyanate recovered from | PU1 | PU2 | CPU1 | CPU2 |
| Isocyanate | MDI | MDI | MDI | TDI |
| Polyol | PTMO | PTMO | PTMO | PTMO |
| Chain extender | BDO | BDO | BDO | BDO |
| Temperature (in ° C.) (T1, T2) | 65, 100 | 65, 85 | 65, 100 | 65, 85 |
| Time Pre-polymer | 25 min | 3 h | 25 min | 3 h |
| Time (in h); Chain extension | 34 | 24 | 34 | 24 |

### Comparative Example 1: Depolymerization Reaction with Lewis Base Only, with the Addition of a Depolymerization Agent

To test if the addition of base alone was sufficient to promote the formation of an isocyanate, the depolymerization agent was omitted and a blank reaction was carried out in toluene at 80° C. For the reaction, PU2 (250 mg) was soaked in 3 mL of toluene to swell the crosslinked polymer, and triethylamine (0.2 mL) was added. The mixture was heated to 80° C. and allowed to react for 2 h, according to an embodiment of the present invention. As expected, ATR-FTIR analysis confirmed absence of isocyanate, evident from the missing peak at ˜2270 cm⁻¹ in the IR spectrum, highlighting the importance of the organoboron agent.

Based on present observation and prior literature, and without wishing to be bound by any particular theory, it is believed that a base-promoted deprotonation of the urethane group, followed by the nucleophilic substitution of Cl by urethane, and a rearrangement within the resulting urethane-chlorocatecholborane complex results in the formation of an isocyanate. It is important to note that the formation of isocyanates from urethanes is challenging owing to the tendency of the highly reactive isocyanate to recombine with the alcohol. However, the depolymerization agent used in the urethane-to-isocyanate functional group transformation acts as a capping agent to the alcohol preventing it from recombining with the isocyanate and for the reaction from going in the reverse direction.

### Comparative Example 2: Depolymerization Reaction with a Depolymerization Agent Only, with the Addition of a Lewis Base

Another control experiment using β-chlorocatecholborane in the absence of the base was performed. Polyurethane CPU1 (500 mg) was added to a round-bottom flask with 10 ml of dry toluene. The mixture was stirred at 20° C. for 12 h. β-chlorocatecholborane (235 mg) in 5 mL of dry toluene was added dropwise to the polymer solution, and the resulting mixture was stirred at 80° C. for 2 h. ATR-FTIR analysis showed the presence of isocyanates. However, the isolated yield, after purification by column chromatography using ethyl acetate/hexanes was <6% (wt. %, based on the amount of the polyurethane (CPU1)).

### Comparative Example 3: Depolymerization Reaction with a Polar Solvent

Another control experiment using β-chlorocatecholborane and base was performed, except DMAc was used as a solvent and the reaction was carried out at 150° C. The amounts of all the reactants were kept same as in Example No. 1. The depolymerization procedure, similar to that used for PU1, in Example No. 1, was used except that DMAc (15 mL) was used as a solvent and the reaction was carried out at 150° C. After the reaction, an aliquot from the depolymerization reaction mixture was analysed by ATR-FTIR spectroscopy and the characteristic peak of the isocyanate at 2270 cm⁻¹ was not observed, which means the depolymerization did not proceed in DMAC.

Although the invention is illustrated and described herein with reference to specific embodiments, the invention is not intended to be limited to the details shown. Rather, various modifications may be made in the details within the scope and range of equivalents of the claims and without departing from the invention. While preferred embodiments of the invention have been shown and

described herein, it will be understood that such embodiments are provided by way of example only. Numerous variations, changes and substitutions will occur to those skilled in the art without departing from the spirit of the invention. Accordingly, it is intended that the appended claims cover all such variations as fall within the spirit and scope of the invention.

1. A method for depolymerizing a polyurethane to provide a regenerated isocyanate compound in a single step, the method comprising contacting the polyurethane with a depolymerization agent in the presence of a solvent and a Lewis base,
wherein the depolymerization agent comprises a halide-containing boron compound, and
wherein the regenerated isocyanate mixture comprises a mixture of a regenerated isocyanate compound and at least one of an adduct of a polyol and the halide-containing boron compound, an adduct of a chain extender and the halide-containing boron compound, a residual amount of the Lewis base, or an acid-base adduct.

2. The method according to claim 1, wherein the polyurethane is a thermoplastic polyurethane or a thermoset polyurethane, as represented by the following structures: 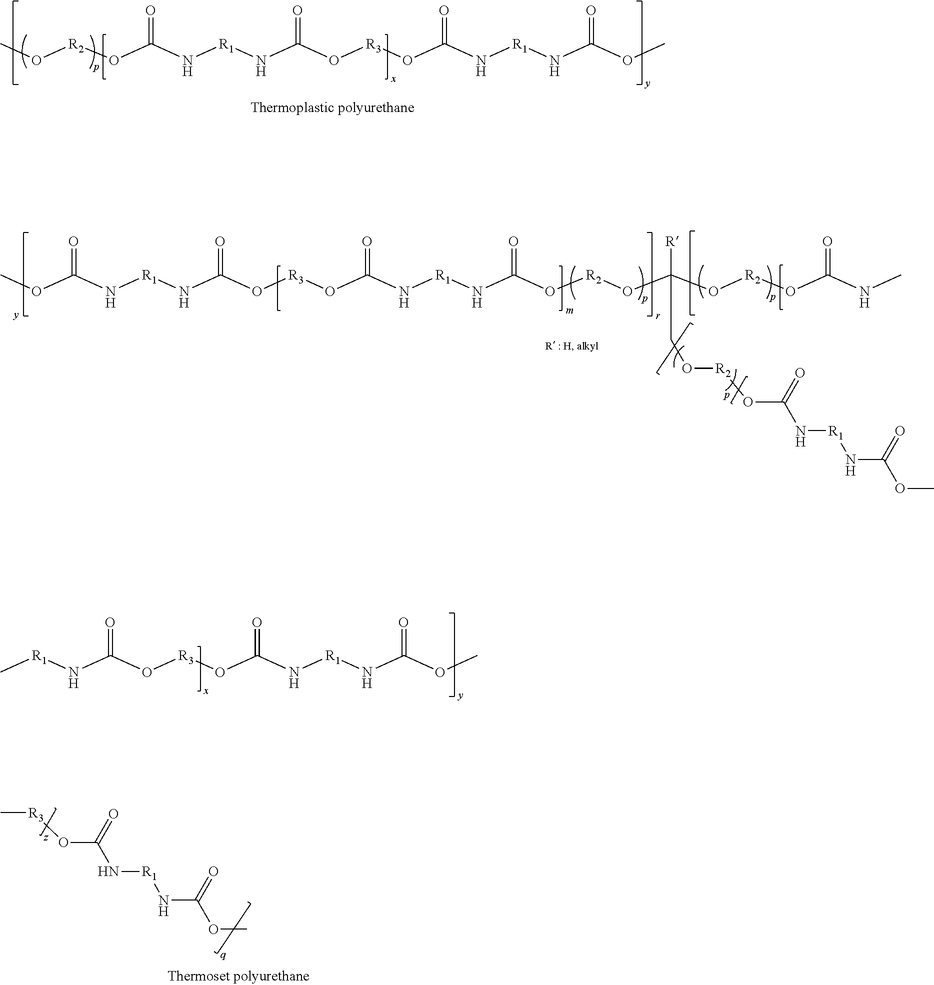
wherein:
R′ is H or a C1-C10 alkyl group;
each R₁ is independently:
(i) an unsubstituted or a substituted alkylene group having 2 to 75 carbon atoms where one or more carbon atoms in the alkylene group may be substituted by oxygen, nitrogen or sulfur atoms with the proviso that only carbon atoms are directly bonded to each of the two NH groups in the polyurethane backbone; or
(ii) an unsubstituted or a substituted aromatic-containing group having 6 to 75 carbon atoms; or
(iii) an unsubstituted or a substituted heteroaromatic-containing group having 4 to 75 carbon atoms; or
(iv) an unsubstituted or a substituted cycloalkyl-containing group having 4 to 75 carbon atoms; or
(v) an unsubstituted or a substituted heterocycloalkyl-containing group having 4 to 75 carbon atoms; or
(vi) combinations thereof;
R₂ and R₃ are independently selected from an alkylene group having at least 2 carbon atoms, where:
(i) one of R₂ or R₃ is an alkylene group having 2 to 5 carbon atoms, and the other of R₂ or R₃ is an oligomeric or a polymeric segment having at least 6 carbon atoms, and
(ii) one or more carbon atoms is optionally substituted by oxygen, nitrogen, or sulfur atoms with the proviso that only carbon atoms are directly bonded to each of the end oxygen atoms in the polyurethane backbone;
x, y, z, m, n, and r, each independently is 1 to 200, and p is 3 to 100, and wherein the depolymerization agent is present in an amount of 0.3 to 3 molar equivalents with respect to urethane linkages.

3. The method according to claim 1, wherein the halide-containing boron compound is selected from a halo-organo-borane compound or a boron trihalide.

4. The method according to claim 3, wherein the halo-organo-borane compound comprises one or more of β-chlorocatecholborane, β-bromocatecholborane, β-fluorocatecholborane, and β-iodocatecholborane.

5. The method according to claim 1, wherein the Lewis base comprises one or more of triethylamine, pyridine, dimethylaminopyridine, triethylenediamine, diisopropylethylamine, other trialkylamines, 1,8-diazabicyclo [5.4.0]undec-7-ene (DBU), 1,5-diazabicyclo[4.3.0]non-5-ene (DBN), amidine derivatives, lithium diisopropylamide, and quinuclidine.

6. The method according to claim 1, wherein the solvent comprises one or more of toluene, hexane, heptane, pentane, benzene, and cyclohexane.

7. The method according to claim 1, wherein the depolymerization is carried out at a temperature in a range of 20° C. to 150° C.

8. The method according to claim 1, wherein the depolymerizing is carried out in an inert environment comprising one or more of nitrogen, argon, helium, and dry air.

9. The method according to claim 1, wherein the polyol comprises poly(tetramethylene oxide), polyethylene glycol, polyethylene glycol trimethylolpropane triether, polypropylene glycol, or combinations thereof, and
wherein the chain extender comprises 1,4-butanediol, glycerol, ethylene glycol, 1,6-hexanediol, or combinations thereof.

10. The method according to claim 1, further comprising isolating recovered isocyanate from the regenerated isocyanate mixture by treatment of the regenerated isocyanate mixture with at least one of chromatography, distillation, differential extraction, or crystallization.

11. A method of forming a polyurethane comprising reacting the isolated recovered isocyanate from the method according to claim 10 with one or more polyols and/or chain extenders in the presence of a polymerization catalyst, and, an optional solvent.

12. The method according to claim 11, wherein the polymerization catalyst comprises a tin-containing catalyst or an amine catalyst.

13. The method according to claim 12, wherein the tin-containing catalyst comprises dibutyltin dilaurate, dioctyltin dilaurate, dicotyl carboxylate, stannous octoate, or mixtures thereof, and
wherein the amine catalyst comprises 1,4-diazabicyclo[2.2.2]octane (DABCO), dimethylethanolamine, triethylenediamine, or mixtures thereof.

14. A composition comprising the polyurethane prepared according to the method of claim 10 and a halide-containing boron compound.

15. The composition of claim 14, wherein the composition further comprises a solvent.

16. The composition according to claim 14, wherein the composition further comprises one or more of a virgin polyurethane, an inert filler, and an additive.

17. The composition according to claim 14, wherein the composition has a bio-based content in the range of 20 to 100%, according to ASTM-D6866.

18. An article comprising the composition according to claim 14.

19. A method comprising:
(i) providing a regenerated isocyanate mixture obtained by the method for depolymerizing according to claim 1;
(ii) purifying the regenerated isocyanate mixture by at least one of chromatography, distillation, differential extraction, or crystallization to obtain an isolated recovered isocyanate compound; and
(iii) forming a recycled polyurethane by reacting the recovered isocyanate compound with one or more polyols in the presence of a catalyst, wherein the one or more polyols comprises a recovered polyol, a bio-derived polyol, a petroleum-based polyol, or a mixture thereof.
